(19) United States
(12) Patent Application Publication
(10) Pub. No.: US 2026/0319449 A1
(43) Pub. Date: Oct. 15, 2026

(54) TOTAL VARIATION ANALOG-TO-DIGITAL CONVERTER FOR ELECTROENCEPHALOGRAM ACQUISITION

(71) Applicant: TIANJIN UNIVERSITY, Tianjin (CN)

(72) Inventors: Biao SUN, Tianjin (CN); Jing YANG, Tianjin (CN); Xiaochen REN, Tianjin (CN); Chi YAO, Tianjin (CN)

(73) Assignee: TIANJIN UNIVERSITY, Tianjin (CN)

(21) Appl. No.: 19/557,011

(22) Filed: Mar. 4, 2026

Related U.S. Application Data
(63) Continuation of application No. PCT/CN2025/103727, filed on Jun. 26, 2025.

(30) Foreign Application Priority Data
Apr. 14, 2025 (CN) 202510461777.6

(51) Int. Cl. G05B 15/02

(52) U.S. Cl. CPC G05B 15/02

(57) ABSTRACT

Firstly, a total variation operator is employed to compute second-order delta encoding, which can more effectively utilize the sparsity of EEG signals and significantly improve the compression ratio of EEG signals. Secondly, a fixed dual-buffer module is designed to enable the generation of second-order delta encoded values in a concise and efficient manner. Finally, a serialized bit stream generator is designed to enable second-order delta data collected from multiple parallel channels to be subjected to serialized packaging. It is not only highly scalable, but also suitable for multi-channel acquisition, and can enable reliable interfacing with a subsequent synchronous clock wireless transmission system, thus avoiding the overhead and latency caused by synchronization-asynchronous interface coordination, and further improving the efficiency of signal acquisition.

### CROSS-REFERENCE TO RELATED APPLICATIONS

This application is a continuation of International Application No. PCT/CN2025/103727, filed on Jun. 26, 2025, which claims priority to Chinese Patent Application No. 202510461777.6, filed on Apr. 14, 2025. All of the aforementioned applications are incorporated herein by reference in their entireties.

### TECHNICAL FIELD

The present disclosure belongs to the field of brain-computer interfaces, and in particular, relates to a total variation analog-to-digital converter (TV-ADC) for electroencephalogram (EEG) acquisition.

### BACKGROUND

A brain-computer interface (BCI), as a cutting-edge technology that connects the human brain with external electronic devices, is spearheading a revolution in the fields of neural therapy, neuroscience research, and human-computer interaction. Through real-time monitoring and analysis of brain activity, the BCI offers hope for patients with severe disabilities (such as paralysis and locked-in syndrome) to regain partial bodily functions. With the aid of BCI technology, patients can control external devices through their thoughts, thereby improving their quality of life. In addition, the BCI also plays a pivotal role in neuroscience research, which not only helps researchers to deeply explore the neurodynamics of the brain, but also facilitates the understanding of neurological disorders and the development of therapeutic approaches. The widespread application of BCIs has driven innovations across multiple domains, ranging from basic neuroscience to clinical treatment.

Among the various sensing technologies for BCIs, electroencephalogram (EEG) has become the most commonly used signal acquisition method due to its advantages such as non-invasiveness, convenience, and high temporal and spatial resolution. EEG can reflect the electrical activity of neuronal populations in the cerebral cortex in real time, and is thus widely used in clinical and scientific research fields. However, with the continuous increase in the number of EEG acquisition channels, the acquisition and transmission of EEG signals are facing unprecedented challenges. Each additional acquisition channel leads to a dramatic surge in the volume of data to be transmitted, which in turn places higher requirements on the transmission bandwidth, energy consumption, and processing capacity of a system.

Wireless data transmission is widely adopted in modern BCI systems to ensure the free movement of users without affecting their daily lives. However, with the rapid increase in data volume, the bandwidth of wireless transmission has become a bottleneck, and how to reduce the volume of transmitted data on the premise of ensuring data integrity has emerged as the key to addressing this issue. Traditional EEG acquisition systems typically adopt architectures combining analog front-end sensors (including preamplification stages) and Nyquist analog-to-digital converters (ADCs), which convert analog signals into digital signals before subsequent processing and analysis. Nevertheless, such traditional architectures suffer from severe energy efficiency issues, which are particularly critical in scenarios where BCIs require long-term operation and frequent data transmission.

Owing to the constraints of wireless data transmission and battery capacity, the conventional Nyquist ADC architecture fails to achieve effective optimization in terms of energy consumption, resulting in high power consumption and transmission latency. Traditional analog-to-digital conversion methods, based on the Nyquist sampling theorem, require sufficiently high sampling rates to accurately capture the frequency information of EEG signals. Given that the frequency spectrum of EEG signals is relatively limited, especially with relatively weak high-frequency components, the traditional Nyquist sampling method tends to generate a large volume of redundant data, which not only increases transmission burden, but also wastes substantial power resources. Furthermore, the sparsity characteristics of EEG signals are not fully exploited.

EEG signals exhibit significant sparsity in temporal variation, i.e., the signals change little over the vast majority of the time and only experience significant fluctuations in a few moments. To address this redundancy issue, researchers have proposed the design of analog-to-digital converters based on signal delta encoding (e.g., Delta-ADCs), aiming to reduce data transmission burden and improve system energy efficiency through compressed sampling. By sampling delta information only when input signals cross a preset threshold, Delta-ADCs can effectively avoid unnecessary data acquisition during signal silent periods, thereby saving energy while still enabling efficient signal recovery.

However, although Delta-ADCs are theoretically capable of significantly improving acquisition efficiency, existing implementations still face several critical issues. Firstly, the delta encoding method adopted by Delta-ADCs is not always superior to the traditional Nyquist sampling-based analog-to-digital converter. In certain application scenarios, especially when signals change frequently or exhibit high complexity, the performance of Delta-ADCs may even be inferior to that of the traditional successive approximation register (SAR) analog-to-digital converters. In addition, the existing Delta-ADCs generally employ first-order difference calculation to generate first-order delta encoding. However, first-order delta encoding does not fully exploit the sparsity of EEG signals, resulting in limited compression effects and failure to fully improve acquisition efficiency. Finally, most of the existing Delta-ADC systems adopt an asynchronous sampling mode, whereas wireless brain-computer interface systems typically use a synchronous digital processing method. Therefore, it leads to considerable overhead and latency in the coordination between system interfaces, which further affects the overall performance and energy efficiency.

It is therefore evident that, to meet the demands of modern BCIs for efficient acquisition, low-power transmission, and high-quality signal recovery, it is crucial to design a novel analog-to-digital converter that can effectively compress EEG signals, improve acquisition efficiency, and adapt to wireless transmission requirements. This design not only needs to fully exploit the sparse nature of EEG signals, but also considers the system's power consumption, sampling efficiency, and compatibility with subsequent processing modules.

### SUMMARY

In order to overcome the problem of low efficiency of EEG signal acquisition in the prior art, the present disclosure provides a TV-ADC for EEG acquisition, which comprehensively improves signal acquisition efficiency while maintaining the simplicity of a circuit topology structure. Firstly, a total variation operator is designed to compute second-order delta encoding, which can make more effective use of the sparsity of EEG signals and significantly improve the compression ratio of EEG signals compared with the existing first-order delta encoding. Secondly, a fixed dual-buffer module is designed to avoid the circuit complexity caused by the need for switching between the existing dual-buffer modules, enabling the generation of second-order delta encoded values in a concise and efficient manner. Finally, a serialized bit stream generator is designed to enable second-order delta data collected from multiple parallel channels to be subjected to serialized packaging. It is not only highly scalable, but also suitable for multi-channel acquisition, and can enable reliable interfacing with a subsequent synchronous clock wireless transmission system, thus avoiding the overhead and latency caused by synchronization-asynchronous interface coordination, and further improving the efficiency of signal acquisition.

The present disclosure relates to a TV-ADC for EEG acquisition, which consists of a multiplexer, a fixed dual-buffer module, a digital-to-analog converter (DAC) controller, a DAC, a comparator, an incremental counter, and a serialized bit stream generator, where the multiplexer is controlled by a global clock CLKADC of the TV-ADC, switches an acquisition channel in each clock cycle and stabilizes the current EEG signals collected to support multi-channel parallel acquisition.

Further, the fixed dual-buffer module is controlled by the global clock CLKADC of the TV-ADC, and is responsible for storing and outputting a previous collected value from each channel to support computation of second-order delta encoding while avoiding the complexity of the traditional dual-buffer structure.

Furthermore, the DAC controller is configured to read the previous collected values of the fixed dual-buffer module and generate DAC control codes in combination with output of the incremental counter.

Furthermore, the DAC is configured to read in the DAC control codes output by the DAC controller, convert the codes into EEG signals to be compared, and store final output of the DAC as a current collected value of the current channel in the fixed dual-buffer module after multiple comparisons.

Furthermore, the comparator is configured to compare the EEG signals to be collected output by the multiplexer with the EEG signals to be compared output by the DAC, and input comparison results into the incremental counter for incremental counting.

Furthermore, the incremental counter is controlled by the global clock CLKADC of the TV-ADC, and is configured to read in an output result of the comparator in each clock cycle, calculate a second-order delta between a current acquired EEG signal and a previous collected value, and transmit the second-order delta to the DAC controller and the serialized bit stream generator.

Furthermore, the serialized bit stream generator is controlled by the global clock CLK ADC of the TV-ADC, and is configured to store and convert a signal delta output by the incremental counter into a bit stream in each clock cycle to implement data compression, perform serialized packaging to ensure synchronous data transmission, and generate a final bit stream output by the TV-ADC.

The present disclosure employs the total variation operator to compute second-order delta encoding, which can make more effective use of the sparsity of EEG signals and significantly improve the compression ratio of EEG signals compared with the existing first-order delta encoding. The fixed dual-buffer module is designed to avoid the circuit complexity caused by the need for switching between the existing dual-buffer modules, enabling the generation of second-order delta encoded values in a concise and efficient manner. A serialized bit stream generator is designed to enable second-order delta data collected from multiple parallel channels to be subjected to serialized packaging. It is not only highly scalable, but also suitable for multi-channel acquisition, and can enable reliable interfacing with a subsequent synchronous clock wireless transmission system, thus avoiding the overhead and latency caused by synchronization-asynchronous interface coordination, and further improving the efficiency of signal acquisition.

### BRIEF DESCRIPTION OF THE DRAWINGS

In order to illustrate the technical solutions in embodiments of the present disclosure more clearly, the following briefly introduces the accompanying drawings required for describing the embodiments or the prior art. Apparently, the accompanying drawings in the following description are only some embodiments of the present disclosure. For those of ordinary skill in the art, other drawings can also be obtained from these drawings without creative efforts.

FIG. 1 shows an overall architecture of a TV-ADC for EEG acquisition proposed by the present disclosure.

FIG. 2 is a schematic diagram of a fixed dual-buffer module.

FIG. 3 is a working principle diagram of a DAC controller.

FIG. 4 is a structural diagram of a DAC.

FIG. 5 is a structural diagram of a comparator.

FIG. 6 is a working principle diagram of an incremental counter.

FIG. 7 is an example timing diagram of the incremental counter.

FIG. 8 is a working principle diagram of a serialized bit stream generator.

FIG. 9 shows data results collected by two types of ADCs.

FIG. 10 shows normalized results of data collected by the two types of ADCs.

FIG. 11 shows results of converting the data acquired by the two types of ADCs into bit streams.

FIG. 12 shows the counts of 0s and 1s in the bit streams, as well as the total count of binary values.

### DETAILED DESCRIPTION OF THE EMBODIMENTS

In the following description, for illustration purposes rather than limitation, specific details such as specific system structures and technologies are provided to facilitate a thorough understanding of the embodiments of the present disclosure. However, those skilled in the art should be aware that the present disclosure may also be implemented in other embodiments without these specific details. In other instances, detailed descriptions of well-known systems, devices, circuits, and methods are omitted to avoid unnecessary details hindering the description of the present disclosure.

For an N-point EEG time series signal X=[x₁, x₂, . . . , x_{N}]^{T}, its total variation delta and total variation operator are defined as:

$$[ \begin{matrix}{d}_{1} \\ {d}_{2} \\ {d}_{3} \\ {d}_{4} \\ ⋮ \\ {d}_{N}\end{matrix} ] = [ \begin{matrix}1 & 0 & 0 & 0 & ⋯ & 0 \\ - 2 & 1 & 0 & 0 & ⋯ & 0 \\ 1 & - 2 & 1 & 0 & ⋯ & 0 \\ 0 & 1 & - 2 & 1 & ⋯ & 0 \\ ⋮ & ⋮ & ⋮ & ⋮ & ⋱ & ⋮ \\ 0 & 0 & 0 & 0 & ⋯ & 1\end{matrix} ] \cdot [ \begin{matrix}{x}_{1} \\ {x}_{2} \\ {x}_{3} \\ {x}_{4} \\ ⋮ \\ {x}_{N}\end{matrix} ]$$

where D=[d₁, d₂, . . . , d_{N}]^{T} denotes the total variation delta, and

$$A = [ \begin{matrix}1 & 0 & 0 & 0 & ⋯ & 0 \\ - 2 & 1 & 0 & 0 & ⋯ & 0 \\ 1 & - 2 & 1 & 0 & ⋯ & 0 \\ 0 & 1 & - 2 & 1 & ⋯ & 0 \\ ⋮ & ⋮ & ⋮ & ⋮ & ⋱ & ⋮ \\ 0 & 0 & 0 & 0 & ⋯ & 1\end{matrix} ]$$

denotes the total variation operator. For each collected data point xₜ, its total variation delta is given by dₜ=xₜ−(2xₜ₋₁−xₜ₋₂). It can thus be seen that calculating the total variation delta of the current collected data point requires the values of the two preceding collected data points. Therefore, the total variation delta is a type of second-order delta, and the total variation operator is a type of second-order difference operator.

To implement second-order delta acquisition, the present disclosure proposes a TV-ADC for EEG acquisition, with an overall architecture as shown in FIG. 1. The TV-ADC consists of a multiplexer, a fixed dual-buffer module, a DAC controller, a DAC, a comparator, an incremental counter, and a serialized bit stream generator.

The multiplexer is controlled by a global clock CLKADC of the TV-ADC, switches an acquisition channel in each clock cycle, and stabilizes the current signals collected after the channel is switched to support multi-channel parallel acquisition. The multiplexer in this circuit adopts a standard time-division multiplexing structure, which allows signals from L channels to share a single physical channel for transmission and to occupy the channel sequentially in different CLKADC cycles, thus achieving efficient data acquisition.

According to the present disclosure, the time-division multiplexing structure is implemented by a multiplexer circuit, and a switch position toggles once per clock cycle to select different acquisition channels. After the multi-channel switch, the signals enter a sample-and-hold circuit, where a switched capacitor circuit is employed to physically hold the signals, which ensures the stability of the signals during the acquisition process, thus guaranteeing the accuracy and reliability of subsequent acquisition.

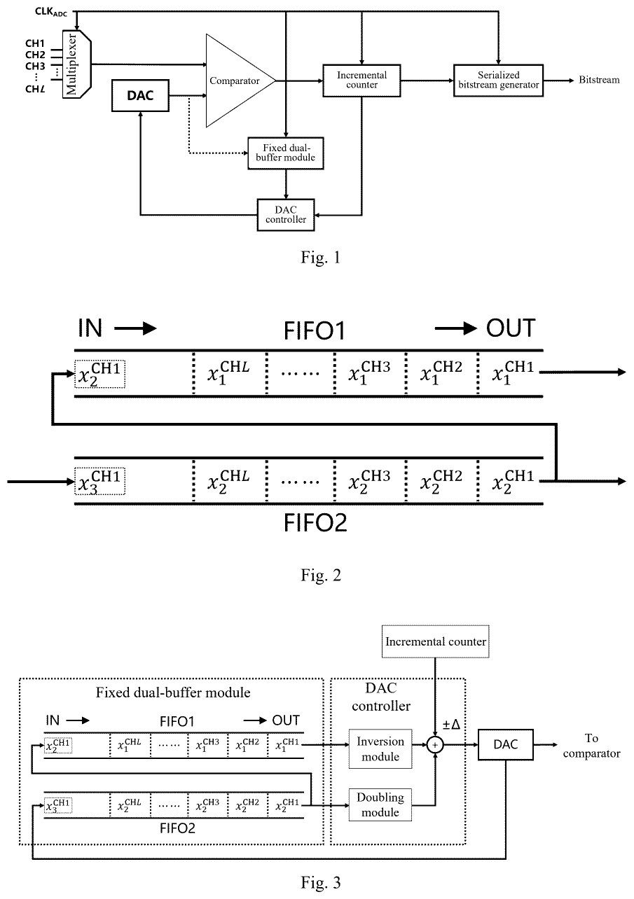

As shown in FIG. 2, the fixed dual-buffer module is controlled by the global clock CLKADC of the TV-ADC, and is responsible for storing and outputting a previous collected value from each channel to support computation of second-order delta encoding while avoiding the complexity of the traditional dual-buffer structure.

Specifically, this module comprises two first-in first-out (FIFO) queues of equal length, where the queue length is correlated with the number of channels from which data is about to be collected. Taking L channels from which data is about to be collected as an example, each FIFO queue needs to store at least L pieces of data. To prevent queue overflow, a certain margin must be reserved, so that the length of each FIFO queue is set to 1.5 L in the present disclosure. The two FIFO queues are arranged side by side, and each FIFO queue can read data independently. The input of FIFO1 can only be obtained from the output of FIFO2, and data cannot be input from the outside; while the input of FIFO2 can directly receive external inputs. It can thus be seen that the fixed dual-buffer module forms a single-input dual-output structure. This structure is designed to cooperate with the subsequent DAC controller and read data in a fixed manner, thereby avoiding the increase in circuit complexity and power consumption caused by frequent switching of readout ports.

As shown in FIG. 3, the DAC controller is configured to read the previous collected values of the fixed dual-buffer module and generate DAC control codes in combination with output of the incremental counter.

Specifically, under the control of the global clock CLK_{ADC}, the TV-ADC sequentially acquires data from the L channels, with the data of one channel being collected per clock cycle. The acquisition is first performed on the first channel, yielding a collected value of

$${x}_{t}^{CH 1} ,$$

and the workflow of the DAC controller is as follows:

Assuming that data stored in the current fixed dual-buffer module are respectively:

$$FIFO 1 = [ \begin{matrix}{x}_{t - 2}^{CHL} & ⋯ & {x}_{t - 2}^{CH 3} & {x}_{t - 2}^{CH 2} & {x}_{t - 2}^{CH 1} ]\end{matrix}$$

$$FIFO 2 = [ \begin{matrix}{x}_{t - 1}^{CHL} & ⋯ & {x}_{t - 1}^{CH 3} & {x}_{t - 1}^{CH 2} & {x}_{t - 1}^{CH 1}\end{matrix} ] .$$

Step 1: extracting previous two collected values

$${x}_{t - 2}^{CH 1}$$

and

$${x}_{t - 1}^{CH 1}$$

of the first channel respectively from FIFO1 and FIFO2 in the fixed dual-buffer module, inputting the collected value

$${x}_{t - 2}^{CH 1}$$

into a doubling module for doubling to obtain

$$- {x}_{t - 2}^{CH 1} ,$$

inputting the collected value

$${x}_{t - 1}^{CH 1}$$

into a doubling module for doubling to obtain

$$2 {x}_{t - 1}^{CH 1} ,$$

and adding the two results together to obtain an initial comparison value

$$2 {x}_{t - 1}^{CH 1} - {x}_{t - 2}^{CH 1}$$

where the inversion module is implemented by an inverter circuit to invert a sign bit of input data and output a result; and the doubling module is implemented by a shift register circuit to shift input data left by 1 bit, thereby achieving a doubling effect.
Step 2: using the incremental counter to input a delta value kΔ and adding the input value to the initial comparison value to obtain a to-be-compared value

$$2 {x}_{t - 1}^{CH 1} - {x}_{t - 2}^{CH 1} + k Δ ,$$

where Δ is a minimum quantization unit of the TV-ADC, and an initial value of the delta value is 0Δ.
Step 3: inputting the to-be-compared

$$2 {x}_{t - 1}^{CH 1} - {x}_{t - 2}^{CH 1} + k Δ$$

into the DAC as a DAC control code, and converting the value into a corresponding signal to be compared.
Step 4: inputting the signal to be compared into the comparator and comparing the signal to be compared with a signal to be collected, where if the signal to be collected is greater than the signal to be compared, the incremental counter is increased by 1Δ, and the delta value becomes (k+1)Δ; and otherwise, the incremental counter is decreased by 1Δ, and the delta value becomes (k−1)Δ.
Step 5: executing steps 2 to 4 cyclically until a difference value between the signal to be collected and the signal to be compared is less than 1Δ, and stopping the cycle at this time, with current output of the DAC being taken as a collected value

$${x}_{t}^{CH 1} .$$

Step 6: inputting

$${x}_{t - 1}^{CH 1}$$

into FIFO1, and inputting

$${x}_{t}^{CH 1}$$

into FIFO2, where data stored in the fixed dual-buffer module are respectively:

$$FIFO 1 = [ \begin{matrix}{x}_{t - 1}^{CH 1} & {x}_{t - 2}^{CHL} & ⋯ & {x}_{t - 2}^{CH 3} & {x}_{t - 2}^{CH 2}\end{matrix} ]$$

$$FIFO 2 = [ \begin{matrix}{x}_{t}^{CH 1} & {x}_{t - 1}^{CHL} & ⋯ & {x}_{t - 1}^{CH 3} & {x}_{t - 1}^{CH 2}\end{matrix} ] .$$

The above process can be followed to continue collecting data from the 2nd to the L-th channels. After data are collected from the L-th channel, the process returns to collecting data from the 1st channel.

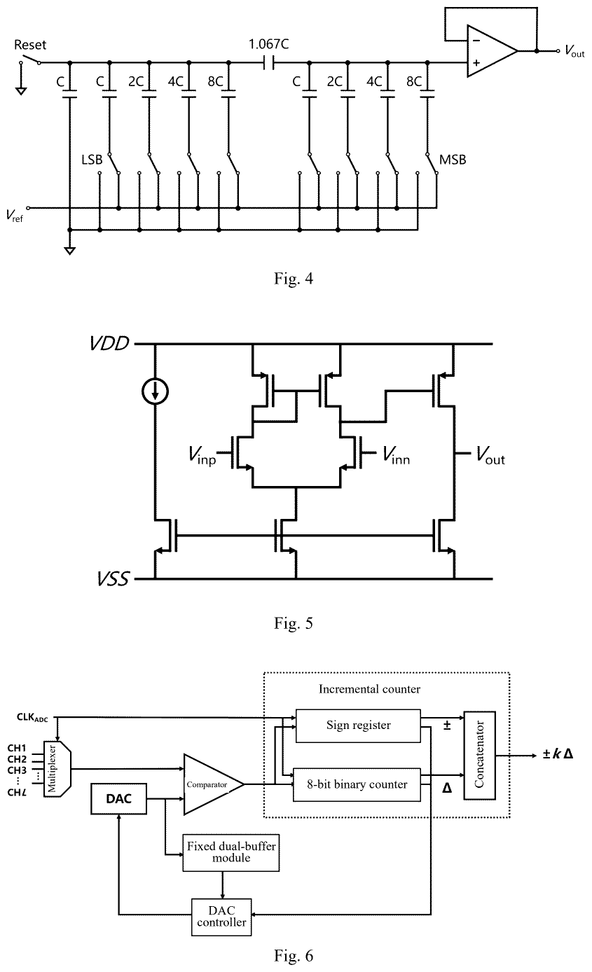

As shown in FIG. 4, the DAC is configured to read in the DAC control codes output by the DAC controller, convert the codes into signals to be compared, and store final output of the DAC as a current collected value of the current channel in the fixed dual-buffer module after multiple comparisons.

Specifically, the DAC is implemented by an 8-bit capacitive voltage-dividing structure, which is composed of an array of 8 weighted capacitors with capacitances of C, 2 C, 4 C, 8 C, C, 2 C, 4 C, and 8 C, respectively. Prior to the start of conversion, a Reset switch is closed to ground upper plates of all capacitors, enabling discharge to the capacitor array. During the conversion phase, the Reset switch is opened, and lower plates of the 8 binary-weighted capacitors are connected to V_{ref} or ground according to the control codes. In addition, a 1.067 C attenuation capacitor is connected to divide the capacitor array into two parts, thereby reducing the types of capacitors. The rightmost side of the DAC constitutes a voltage follower structure through an operational amplifier with a negative feedback connected, which isolates the capacitor array from the output of the DAC to reduce output impedance and enhance load-bearing capacity.

As shown in FIG. 5, the comparator is configured to compare the signals to be collected output by the multiplexer with the signals to be compared output by the DAC, and input comparison results into the incremental counter for incremental counting. To reduce power consumption, the TV-ADC employs a dynamic comparator. The signal to be collected is input through a Vᵢₙₚ terminal, the signal to be compared is input through a Vᵢₙₙ terminal, and the comparison result is output through a Vₒᵤₜ terminal.

As shown in FIG. 6, the incremental counter is controlled by the global clock CLK_{ADC} of the TV-ADC, and is configured to read in an output result of the comparator in each clock cycle, calculate a second-order delta between a current acquired EEG signal and a previous collected value, and transmit the second-order delta to the DAC controller and the serialized bit stream generator.

Specifically, the incremental counter includes two modules, where a sign register is composed of a 1-bit register circuit and is configured to store signs of delta values, and an 8-bit binary counter is composed of eight D flip-flops and is configured to store amplitudes of the delta values.

The incremental counter works as follows: an initial value of the delta values is 0Δ; when output of the comparator is positive, it is determined that an EEG signal to be collected is greater than an EEG signal to be compared, then the incremental counter is increased by 1Δ, the sign register records a positive sign, and the delta value becomes (0+1)Δ; when the output of the comparator is negative, it is determined that the EEG signal to be collected is smaller than the EEG signal to be compared, then the incremental counter is decreased by 1Δ, the sign register records a negative sign, and the delta value becomes (0-1)Δ; after each determination, the delta value is output to the DAC controller for generating a DAC control code; and the above determination steps are performed cyclically until a difference value between the EEG signal to be collected and the EEG signal to be compared is less than 1Δ, at which point the cycle is stopped, and the signs in the sign register are spliced with the values in the 8-bit binary counter to obtain a final second-order delta value.

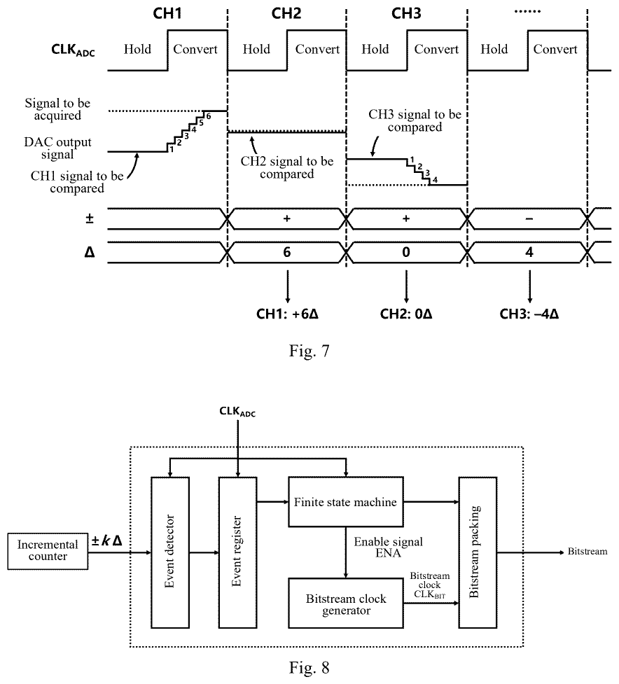

As shown in FIG. 7, this is an example timing diagram of the incremental counter. During each cycle of the global clock CLK_{ADC}, data from one channel is collected. During each clock cycle, the output of the multiplexer remains stable, allowing the incremental counter to perform analog-to-digital conversion.

During the first cycle, the incremental counter performs analog-to-digital conversion on a channel CH1. During the low-level period of CLK_{ADC}, both the EEG signal to be collected and the signal output by the DAC remain stable. At this point, the signal output by the DAC is the EEG signal to be compared for the channel CH1. During the high-level period of CLK_{ADC}, the incremental counter begins analog-to-digital conversion. The first comparison reveals that the EEG signal to be compared is smaller than the EEG signal to be collected, so that the delta is increased by 1Δ and the comparison continues; and after six increments of 1Δ, the difference between the EEG signal to be compared and the EEG signal to be collected is less than 1Δ, indicating that the conversion is complete. At the start of the next CLK_{ADC} cycle, the incremental counter outputs the delta value of the channel CH1, which is +6Δ.

During the second cycle, the incremental counter performs analog-to-digital conversion on a channel CH2. During the low-level period of CLK_{ADC}, both the EEG signal to be collected and the signal output by the DAC remain stable. At this point, the signal output by the DAC is the EEG signal to be compared for the channel CH2. During the high-level period of CLK_{ADC}, the incremental counter begins analog-to-digital conversion. The first comparison reveals that the difference between the EEG signal to be compared and the EEG signal to be collected is less than 1Δ, indicating that the conversion is complete. At the start of the next CLK_{ADC} cycle, the incremental counter outputs the delta value of the channel CH2, which is 0Δ. At this point, it is determined that there is no delta between the current collected signal from the channel CH2 and the previous collected signal therefrom.

During the third cycle, the incremental counter performs analog-to-digital conversion on a channel CH3. During the low-level period of CLK_{ADC}, both the EEG signal to be collected and the signal output by the DAC remain stable. At this point, the signal output by the DAC is the EEG signal to be compared for the channel CH3. During the high-level period of CLK_{ADC}, the incremental counter begins analog-to-digital conversion. The first comparison reveals that the EEG signal to be compared is greater than the EEG signal to be collected, so that the delta is decreased by 1Δ and the comparison continues; and after four decrements of 1Δ, the difference between the EEG signal to be compared and the EEG signal to be collected is less than 1Δ, indicating that the conversion is complete. At the start of the next CLK_{ADC} cycle, the incremental counter outputs the delta value of the channel CH3, which is −4Δ.

The other channels perform analog-to-digital conversion cycle by cycle in accordance with the aforementioned conversion rules.

As shown in FIG. 8, the serialized bit stream generator is controlled by the global clock CLK_{ADC} of the TV-ADC, and is configured to store and convert a signal delta output by the incremental counter into a bit stream in each clock cycle to implement data compression, perform serialized packaging to ensure synchronous data transmission, and generate a final bit stream output by the TV-ADC.

Specifically, the serialized bit stream generator at the rising edge of each global clock CLK_{ADC} causes the event judge to read the delta value from the incremental counter and determine whether the delta value constitutes a second-order delta event; when the delta value is not equal to 0Δ, the second-order delta event is determined to be formed, and the delta value is stored in an event register.

Each second-order delta event requires 9+log₂L bits of storage space, where log₂L bits are configured for numbering of the L channels, 1 bit is configured to store the signs of the delta values, and 8 bits are configured to store the amplitudes of the delta values. In addition, the system is equipped with a 9+log₂L-bit replica memory for parallel data caching and processing to improve data throughput efficiency.

After all L channels complete one collection each, a complete data acquisition frame is formed. Each time an acquisition frame is completed, a finite state machine generates an enable signal ENA and inputs the signal to a bit stream clock generator. The clock generator has a built-in ring oscillator, which is specially configured to provide a clock CLK_{BIT} for bit stream packaging.

Subsequently, the finite state machine scans the data in the event register and performs bit stream packaging based on the clock CLK_{BIT}, and a TV-ADC data bit stream is finally generated.

The TV-ADC proposed in the present disclosure is compared with a current state-of-the-art Nyquist ADC. The experimental setup is as follows: a subject wears an EEG cap and sits quietly in front of a computer, and two electrode channels are led out of the EEG cap, where the channel 1 is used for data acquisition with the Nyquist ADC, and the channel 2 is used for data acquisition with the TV-ADC. The acquired data is transmitted to the computer via wired communication for subsequent analysis.

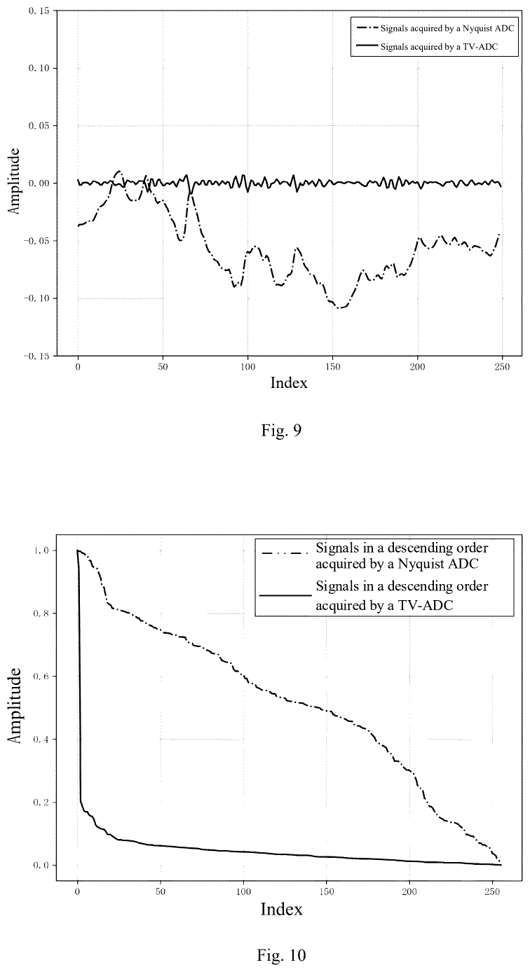

The data acquired by the two types of ADCs is illustrated in FIG. 9. It can be observed that the data acquired by the Nyquist ADC exhibits large fluctuations and high amplitude, whereas the data acquired by the TV-ADC shows small fluctuations, and most of the acquired values are very close to 0, with only a few values being relatively large, thus demonstrating high sparsity.

The data collected by the two types of ADCs are arranged in a descending order and normalized, as illustrated in FIG. 10. It can be seen that the sparsity of the data collected by the TV-ADC is significantly higher than that of the data collected by the Nyquist ADC.

The data collected by the two types of ADCs are converted into bit streams, as illustrated in FIG. 11. It can be seen that the bit stream of the TV-ADC is significantly sparser than that of the Nyquist ADC.

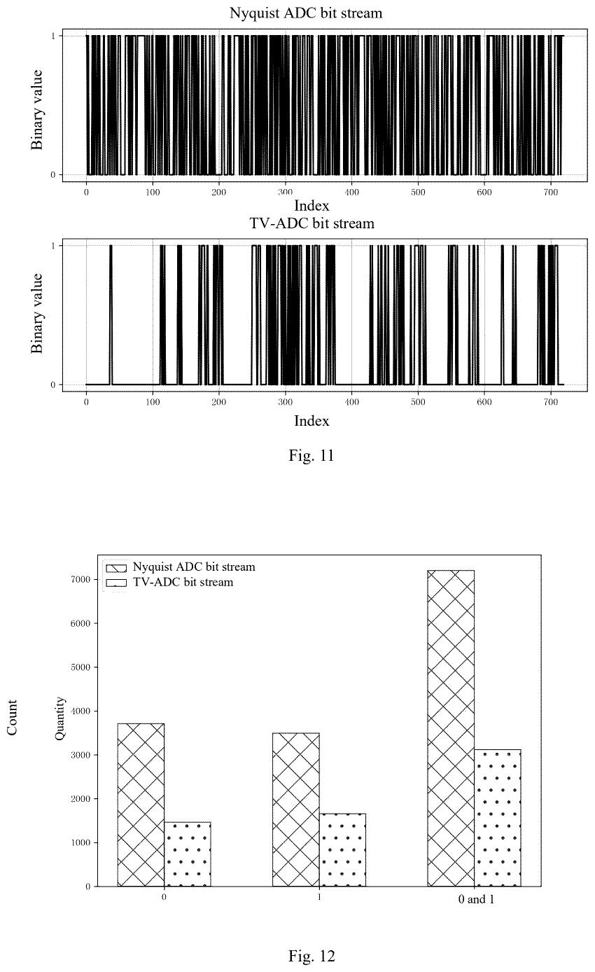

The data acquired by the two types of ADCs are converted into bit streams, and the counts of 0s and 1s in the bit streams as well as the total count of binary values are calculated, as illustrated in FIG. 12. It can be concluded that the data volume of the TV-ADC bit stream accounts for 39.7% of that of the Nyquist ADC bit stream.

The above analysis indicates that the data volume required to be transmitted by the TV-ADC is significantly lower than that required to be transmitted by the Nyquist ADC, thus achieving higher energy efficiency.

It should be understood that the sequence numbers of the steps in the foregoing embodiments do not imply the order of execution; and instead, the execution order of each process shall be determined by its function and inherent logic, and shall not constitute any limitation on the implementation process of the embodiments of the present disclosure.

Those skilled in the art can clearly understand that, for the convenience and conciseness of description, the foregoing division of functional units and modules is provided merely as an example for illustration. In practical applications, the aforementioned functions may be allocated to different functional units and modules for completion as required; and in other words, an internal structure of the apparatus may be divided into different functional units or modules to implement all or part of the functions described above. The various functional units and modules in the embodiments may be integrated into a single processing unit, or each unit may exist physically alone, or two or more units may be integrated into one unit. The above integrated units may be implemented either in the form of hardware or in the form of software functional units. In addition, the specific names of the respective functional units and modules are merely for the convenience of mutual distinction, and shall not be used to limit the protection scope of the present application. For the specific working processes of the units and modules in the above-mentioned system, reference may be made to the corresponding processes in the foregoing method embodiments, which will not be repeated herein.

In the foregoing embodiments, the description of each embodiment has its own emphasis, and for a part that is not described in detail or recorded in a certain embodiment, reference may be made to related descriptions of other embodiments.

Those of ordinary skill in the art may be aware that, in combination with the examples described in the embodiments disclosed herein, units and algorithm steps can be implemented by electronic hardware or a combination of computer software and electronic hardware. Whether these functions are executed in a hardware or software manner depends on specific applications and design constraints of the technical solutions. Those skilled in the art may use different methods to implement the described functions for all specific applications, but it should not be considered that the implementation goes beyond the scope of the present disclosure.

In the embodiments provided by the present disclosure, it should be understood that the disclosed apparatus/terminal device and method may be implemented in other manners. For example, the apparatus/terminal device embodiments described above are merely illustrative; and for instance, the division of the modules or units is merely a logical function division, and in actual implementation, there may be other division manners. For example, multiple units or components may be combined or integrated into another system, or some features may be omitted or not executed. On the other hand, the mutual coupling, direct coupling or communication connection shown or discussed may be indirect coupling or communication connection via some interfaces, apparatus or units, and may be in electrical, mechanical or other forms.

The units described as separate components may or may not be physically separated; the components shown as units may or may not be physical units, i.e., they may be located in one place, or may also be distributed across multiple network units. Some or all of the units may be selected according to actual needs to achieve the objectives of the solutions in the present embodiments.

In addition, the functional units in the various embodiments of the present disclosure may be integrated into a single processing unit, or each unit may exist physically alone, or two or more units may be integrated into one unit. The above integrated units may be implemented either in the form of hardware or in the form of software functional units.

If the integrated modules/units are implemented in the form of software functional units and sold or used as independent products, they may be stored in a computer-readable storage medium. Based on this understanding, the entire or partial processes in the methods of the foregoing embodiments of the present disclosure may also be accomplished by instructing relevant hardware through computer programs, the computer programs may be stored in a computer-readable storage medium, and when executed by a processor, the computer programs can implement the steps of the various method embodiments described above. The computer programs include computer program codes, and the computer program codes may be in a source code form, an object code form, an executable file form, or certain intermediate forms. The computer-readable medium may include any entity or device capable of carrying the computer program codes, recording media, universal serial bus (USB) flash drives, mobile hard disk drives, magnetic disks, optical disks, a computer memory, a read-only memory (ROM), a random access memory (RAM), electrical carrier signals, telecommunication signals, and software distribution media. It should be noted that the content contained in the computer-readable medium may be appropriately added or deleted according to the requirements of legislation and patent practice in the jurisdiction; and for example, in certain jurisdictions, electrical carrier signals and telecommunication signals are not included in computer-readable media in accordance with legislation and patent practice.

The embodiments described above are merely used to illustrate the technical solutions of the present disclosure rather than limit them. Although the present disclosure has been described in detail with reference to the aforementioned embodiments, those of ordinary skill in the art should understand that they may still modify the technical solutions contained in the aforementioned embodiments or equivalently replace some of the technical features therein. Such modifications or replacements do not cause the essence of the corresponding technical solutions to depart from the spirit and scope of the technical solutions of the various embodiments of the present disclosure, and all of them shall be included within the protection scope of the present disclosure.

1. A total variation analog-to-digital converter (TV-ADC) for electroencephalogram acquisition, wherein the TV-ADC comprises a multiplexer, a fixed dual-buffer module, a digital-to-analog converter controller, a DAC, a comparator, an incremental counter, and a serialized bit stream generator, and the TV-ADC is configured to collect electroencephalogram signals;
the multiplexer, the fixed dual-buffer module, the incremental counter, and the serialized bit stream generator are all controlled by a global clock CLK_{ADC} of the TV-ADC;
the multiplexer switches an acquisition channel in each clock cycle and stabilizes the current electroencephalogram signals collected to support multi-channel parallel acquisition;
the fixed dual-buffer module is responsible for storing and outputting a previous collected value from each channel to support computation of second-order delta encoding;
the DAC controller is configured to read the previous collected values of the fixed dual-buffer module and generate DAC control codes in combination with output of the incremental counter;
the DAC is configured to read in the DAC control codes output by the DAC controller, convert the codes into electroencephalogram signals to be compared, and store final output of the DAC as a current collected value of the current channel in the fixed dual-buffer module after multiple comparisons;
the comparator is configured to compare the electroencephalogram signals to be collected output by the multiplexer with the electroencephalogram signals to be compared output by the DAC, and input comparison results into the incremental counter for incremental counting;
the incremental counter is configured to read in an output result of the comparator in each clock cycle, calculate a second-order delta between a current acquired electroencephalogram signal and a previous collected value, and transmit the second-order delta to the DAC controller and the serialized bit stream generator; and
the serialized bit stream generator is configured to store and convert a signal delta output by the incremental counter into a bit stream in each clock cycle, and generate a final bit stream output by the TV-ADC.

2. The total variation analog-to-digital converter according to claim 1, wherein the multiplexer adopts a standard time-division multiplexing structure, the time-division multiplexing structure is implemented by a multiplexer circuit, and a switch position toggles once per clock cycle to select different acquisition channels.

3. The total variation analog-to-digital converter according to claim 1, wherein the DAC controller comprises an inversion module and a doubling module; the inversion module is implemented by an inverter circuit to invert a sign bit of input data and output a result; and the doubling module is implemented by a shift register circuit to shift input data left by 1 bit, thereby achieving a doubling effect.

4. The total variation analog-to-digital converter according to claim 3, wherein the DAC controller operates as follows:
assuming that data stored in the current fixed dual-buffer module are respectively:
$$FIFO 1 = [ \begin{matrix}{x}_{t - 2}^{CHL} & ⋯ & {x}_{t - 2}^{CH 3} & {x}_{t - 2}^{CH 2} & {x}_{t - 2}^{CH 1}\end{matrix} ]$$
$$FIFO 2 = [ \begin{matrix}{x}_{t - 1}^{CHL} & ⋯ & {x}_{t - 1}^{CH 3} & {x}_{t - 1}^{CH 2} & {x}_{t - 1}^{CH 1}\end{matrix} ]$$
step 1: extracting previous two collected values
$${x}_{t - 2}^{CH 1}$$
and
$${x}_{t - 1}^{CH 1}$$
of a first channel respectively from FIFO1 and FIFO2 in the fixed dual-buffer module, inputting the collected value
$${x}_{t - 2}^{CH 1}$$
into the inversion module for negation to obtain
$$- {x}_{t - 2}^{CH 1} ,$$
inputting the collected value
$${x}_{t - 1}^{CH 1}$$
into the doubling module for doubling to obtain
$$2 {x}_{t - 1}^{CH 1} ,$$
and adding the two results together to obtain an initial comparison value
$$2 {x}_{t - 1}^{CH 1} - {x}_{t - 2}^{CH 1} ;$$
step 2: using the incremental counter to input a delta value kΔ and adding the input value to the initial comparison value to obtain a to-be-compared value
$$2 {x}_{t - 1}^{CH 1} - {x}_{t - 2}^{CH 1} + k Δ ,$$
wherein Δ is a minimum quantization unit of the TV-ADC, and an initial value of the delta value is 0Δ;
step 3: inputting the to-be-compared value
$$2 {x}_{t - 1}^{CH 1} - {x}_{t - 2}^{CH 1} + k Δ$$
into the DAC as a DAC control code, and converting the value into a corresponding signal to be compared;
step 4: inputting the signal to be compared into the comparator and comparing the signal to be compared with a signal to be collected, wherein if the signal to be collected is greater than the signal to be compared, the incremental counter is increased by 1Δ, and the delta value becomes (k+1)Δ; and otherwise, the incremental counter is decreased by 1Δ, and the delta value becomes (k−1)Δ;
step 5: executing steps 2 to 4 cyclically until a difference value between the signal to be collected and the signal to be compared is less than 1Δ, and stopping the cycle at this time, with current output of the DAC being taken as a collected value
$${x}_{t}^{CH 1} ;$$
and
step 6: inputting
$${x}_{t - 1}^{CH 1}$$
into FIFO1, and inputting
$${x}_{t}^{CH 1}$$
into FIFO2, wherein data stored in the fixed dual-buffer module are respectively:
$$FIFO 1 = [ \begin{matrix}{x}_{t - 1}^{CH 1} & {x}_{t - 2}^{CHL} & ⋯ & {x}_{t - 2}^{CH 3} & {x}_{t - 2}^{CH 2}\end{matrix} ]$$
$$FIFO 2 = [ \begin{matrix}{x}_{t}^{CH 1} & {x}_{t - 1}^{CHL} & ⋯ & {x}_{t - 1}^{CH 3} & {x}_{t - 1}^{CH 2}\end{matrix} ] .$$

5. The total variation analog-to-digital converter according to claim 1, wherein the DAC is implemented by an 8-bit capacitive voltage-dividing structure; and the comparator employs a dynamic comparator.

6. The total variation analog-to-digital converter according to claim 1, wherein the incremental counter comprises two modules: a sign register and an 8-bit binary counter, wherein the sign register comprises a 1-bit register circuit and is configured to store signs of delta values, and the 8-bit binary counter comprises eight D flip-flops and is configured to store amplitudes of the delta values.

7. The total variation analog-to-digital converter according to claim 6, wherein the incremental counter works as follows: an initial value of the delta values is 0Δ; when output of the comparator is positive, it is determined that an electroencephalogram signal to be collected is greater than an electroencephalogram signal to be compared, then the incremental counter is increased by 1Δ, the sign register records a positive sign, and the delta value becomes (0+1)Δ; when the output of the comparator is negative, it is determined that the electroencephalogram signal to be collected is smaller than the electroencephalogram signal to be compared, then the incremental counter is decreased by 1Δ, the sign register records a negative sign, and the delta value becomes (0-1)Δ; after each determination, the delta value is output to the DAC controller for generating a DAC control code; and the above determination steps are performed cyclically until a difference value between the electroencephalogram signal to be collected and the electroencephalogram signal to be compared is less than 1Δ, at which point the cycle is stopped, and the signs in the sign register are spliced with the values in the 8-bit binary counter to obtain a final second-order delta value.

8. The total variation analog-to-digital converter according to claim 1, wherein the serialized bit stream generator comprises an event judge, an event register, a finite state machine, a bit stream clock generator, and bit stream packaging.

9. The total variation analog-to-digital converter according to claim 8, wherein the serialized bit stream generator at the rising edge of each global clock CLK_{ADC} causes the event judge to read the delta value from the incremental counter and determine whether the delta value constitutes a second-order delta event; when the delta value is not equal to 0Δ, the second-order delta event is determined to be formed, and the delta value is stored in the event register;
after all L channels complete one collection each, a complete data acquisition frame is formed; each time an acquisition frame is completed, the finite state machine generates an enable signal ENA and inputs the signal to the bit stream clock generator; the clock generator has a built-in ring oscillator, which is specially configured to provide a clock CLK_{BIT} for bit stream packaging; and
the finite state machine scans the data in the event register and performs bit stream packaging based on the clock CLK_{BIT}, and a TV-ADC data bit stream is finally generated.

10. The total variation analog-to-digital converter according to claim 9, wherein in the serialized bit stream generator, each second-order delta event requires 9+log₂L bits of storage space, wherein log₂L bits are configured for numbering of the L channels, 1 bit is configured to store the signs of the delta values, and 8 bits are configured to store the amplitudes of the delta values.
